(19) United States
(12) Patent Application Publication
(10) Pub. No.: US 2011/0029028 A1
(43) Pub. Date: Feb. 3, 2011

(54) MACHINING OF ENCLOSURES FOR IMPLANTABLE MEDICAL DEVICES

(75) Inventors: Charles E. Peters, Blaine, MN (US); Steven T. Deininger, Blaine, MN (US); Michael J. Baade, Zimmerman, MN (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 12/847,830

(22) Filed: Jul. 30, 2010

Related U.S. Application Data
(60) Provisional application No. 61/230,549, filed on Jul. 31, 2009.

(51) Int. Cl. A61N 1/375; B23H 1/00; B23K 11/00

(52) U.S. Cl. 607/2; 219/69.17; 219/78.01

(57) ABSTRACT

Enclosures for implantable medical devices are machined from biocompatible materials using processes such as electric discharge machining and/or milling. Material is machined to create an enclosure. The enclosure may include an enclosure sleeve that has top and bottom caps added where the enclosure sleeve is machined either as a whole or as two separate halves that are subsequently joined together. During construction, circuitry is installed and where the enclosure includes an enclosure sleeve, the open top and bottom may be closed by caps while a connector block module may be mounted to the complete enclosure. The machining process allows materials that are typically difficult to stamp, such as grade 5 and 9 titanium and 811 titanium, that are beneficial to telemetry and recharging features of an implantable medical device to be used while allowing for an enclosure with a relatively detailed geometry and relatively tight tolerances.

### CROSS-REFERENCE TO RELATED APPLICATIONS

The present application claims priority to U.S. Provisional Patent Application No. 61/230,549, filed Jul. 31, 2009, which application is hereby incorporated by reference as if re-written in its entirety.

### TECHNICAL FIELD

Embodiments relate to enclosures for implantable medical devices. More particularly, embodiments relate to machining of the enclosures.

### BACKGROUND

Implantable medical devices include an enclosure that houses the internal circuitry and other components that may be present. The enclosure may be constructed of a biocompatible material such as titanium which provides protection of the circuitry and other components. The enclosure may be hermetically sealed to prevent biological fluids from entering the enclosure and damaging the contents.

The enclosure is typically made of a material such as grade 1 titanium or other biocompatible materials of a similar hardness. Grade 1 titanium may be formed into the enclosure using stamping, where two enclosure halves are stamped from sheet stock material and are then welded together or otherwise attached to form the enclosure. While this grade 1 titanium enclosure provides adequate protection, the enclosure is less than ideal for implantable medical devices with features such wireless recharging and telemetry.

To accommodate these features, other grades of titanium have been used, such as grade 5 and grade 9 titanium as well as 811 titanium. These other grades of titanium typically cause less attenuation of recharging energy being directed into the enclosure and telemetry energy being directed into and out of the enclosure. However, grade 5 and 9 titanium as well as 811 titanium have a hardness or other characteristic that presents problems for the stamping process being used to form the two halves. This material may exhibit springback upon being formed which may cause the halves to exceed allowable dimension tolerances. Furthermore, the geometry that is available is restricted because relatively small radii, intricate details, and deep draws are difficult to achieve.

### SUMMARY

Embodiments address issues such as these and others by providing for machining of enclosures using processes such as electric discharge machining and/or milling. Material is machined to create an enclosure, such as by machining an enclosure sleeve as a whole or by machining halves that may be attached at the sides. For the enclosure sleeve, circuitry is installed and the open top and bottom may be closed by caps while a connector block module may be mounted to the complete enclosure. The machining may be performed on various biocompatible materials including various grades of titanium including grade 5 and grade 9 titanium and 811 titanium.

### DESCRIPTION OF THE DRAWINGS

FIG. 1A shows an electric discharge process for machining at least a portion of an enclosure for an implantable medical device.

FIG. 1B shows a milling process for machining at least a portion of an enclosure for an implantable medical device.

FIG. 2 shows an example of a machined enclosure for an implantable medical device.

FIG. 3A is a perspective view of a machined enclosure half.

FIG. 3B is a perspective view of a machined enclosure half that attaches to the machined enclosure half of FIG. 3A to form an enclosure sleeve.

FIG. 4A is a top view of the machined enclosure half of FIG. 3A.

FIG. 4B is a top cross-sectional view of the machined enclosure half of FIG. 3B.

FIGS. 5A-5D are exploded perspective views of an illustrative embodiment of an implantable medical device.

FIG. 6 shows one example of a set of manufacturing operations to produce an implantable medical device.

FIG. 7 shows another example of a set of manufacturing operations to produce an implantable medical device.

FIG. 8 shows another example of a set of manufacturing operations to produce an implantable medical device.

### DETAILED DESCRIPTION

Embodiments provide for enclosures of implantable medical devices that are machined rather than stamped. The materials chosen for the enclosure may be harder than those materials used in a stamping process while maintaining geometries and tolerances adequate for the implantable medical device. The harder materials used in the machined enclosure may offer better performance for telemetry and recharging.

Machining of the enclosures may be done in various ways. For instance, machining may involve one or more forms of electric discharge machining (EDM), with wire EDM being particularly well suited to the machining of an enclosure sleeve as discussed below. Milling is another example of machining that may be done, alone or in combination with one or more forms of EDM. Other examples of machining are also applicable such as water jetting.

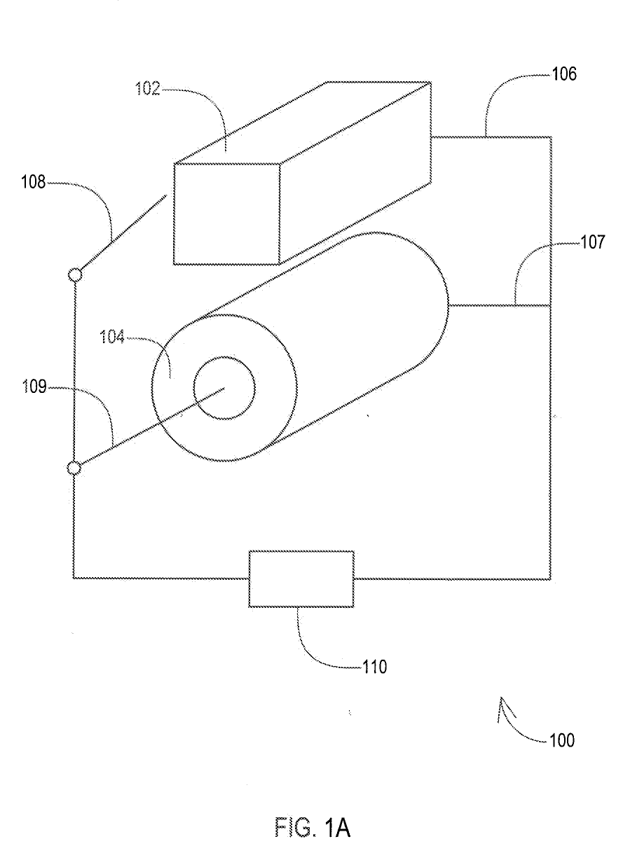

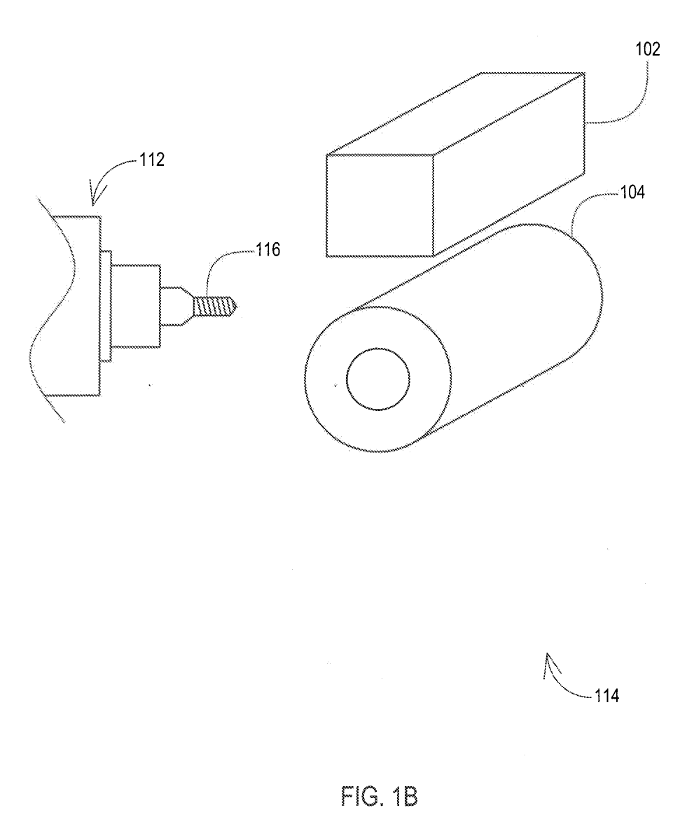

FIG. 1A shows an example of an electric discharge machining EDM process 100 that may be used according to various embodiments. This particular example employs wire EDM which may provide the ability to produce relatively tight radii and relatively detailed geometries, while wall thickness may be maintained at a uniform thickness or may be varied by design. The nature of the wire EDM process 100 dictates that an enclosure sleeve, or halves of an enclosure sleeve, be produced where the top and bottom are open. As discussed below, caps can then be attached to the enclosure sleeve to seal the top and bottom openings of the enclosure sleeve.

In this wire EDM example of machining, the initial workpiece may be of various forms. Two examples of workpieces are shown, a piece of bar stock material 102 and a piece of tubular stock material 104. The wire EDM process 100 may begin with either type of workpiece as well as others. The tubular workpiece 104 is particularly well suited to a wire EDM process where the enclosure is being machined as a whole. Considering the tubular workpiece 104 already has a hollow center where a wire 109 of the wire EDM process may be positioned, the inside geometry of the enclosure can be machined using the wire 109. For a bar workpiece 102, if the enclosure is to be wire EDM machined as a whole, then a hole must first be created within the bar workpiece 102 to allow placement of a wire 108 of the wire EDM so that the inside geometry can be machined using the wire 108.

The wire EDM process 100 uses an electrical power source 110 which applies a voltage potential between the wire 108/109 and an electrical contact 106/107 to the workpiece 102/104. The workpiece 102/104 is present within a dielectric bath. The repeated discharge from the wire 108/109 to the workpiece 102/104 repeatedly removes matter from the workpiece 102/104 to essentially provide a cutting effect. This cutting effect works even in the harder materials such as grade 5 titanium as well as in grade 9 titanium and 811 titanium and does not work harden the material such that an additional annealing step is not needed afterwards when wire EDM is used for the entire enclosure. The wire EDM process 100 may employ a variety of machining wires, including those having a diameter on the order of one ten-thousandth of an inch. Furthermore, a variety of power settings and speeds may be utilized for the wire EDM process 100, with slower speeds generally resulting in smoother surface finishes.

In some embodiments, the wire EDM process 100 may be used to machine the entire enclosure sleeve. In other embodiments, the wire EDM process 100 may be used for only a portion of the enclosure sleeve geometry, such as only the inside geometry, while another machining process such as another form of EDM or milling is used to create the outside or other remaining geometry.

FIG. 1B shows the milling process 114. Here a milling machine 112 includes a milling tool 116. This milling tool 116 is spun at a high angular velocity and brought into contact with the workpiece 102/104 to machine it to the appropriate geometry. One consequence of using milling for at least a portion of the enclosure geometry is that the workpiece 102/104 is work hardened. To account for this, the workpiece 102/104 once milled can be annealed.

In some embodiments, the milling process 114 may be used to machine the entire enclosure, whether in the form of a whole sleeve, enclosure sleeve halves with top and bottom caps, or as non-sleeve enclosure halves of conventional shape. In other embodiments, the milling process 114 may be used for only a portion of the enclosure sleeve geometry, such as only the outside geometry, while another machining process such as wire EDM is used to create the inside or other remaining geometry.

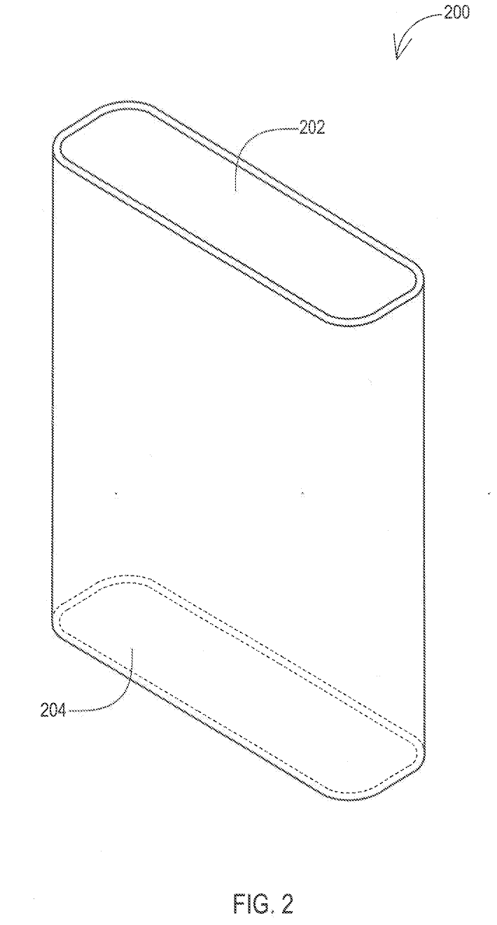

FIG. 2 shows an example of a resulting enclosure sleeve 200 that has been machined as a whole according to various embodiments. The enclosure sleeve 200 includes an open top 202 and bottom 204 which may be capped during subsequent manufacturing steps once circuitry, desiccant, and the like are placed into the enclosure sleeve 200.

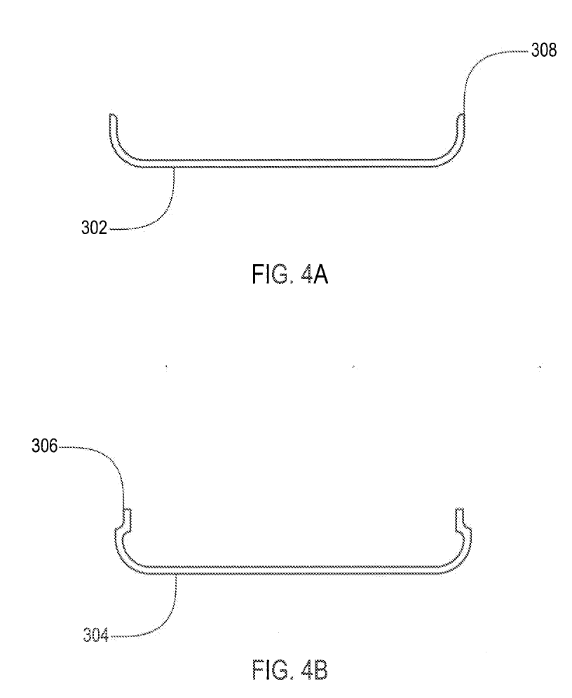

The enclosure sleeve 200 is shown with a particular symmetrical racetrack cross-section that is consistent from top to bottom. It will be appreciated that other cross-sections are applicable as well and that variations in the cross-section from top to bottom are also applicable. For instance, the wall thickness may vary at certain locations by design, which is a direct benefit of machining versus stamping. The wall thickness of the enclosure sleeve 200 may be machined to relatively thin amounts, such as 0.008 inch having a tolerance of 0.001 inch. Machining allows for other small details, such as a radiused edge 308, as shown in FIG. 4A, with a radius on the order of 0.008 inch.

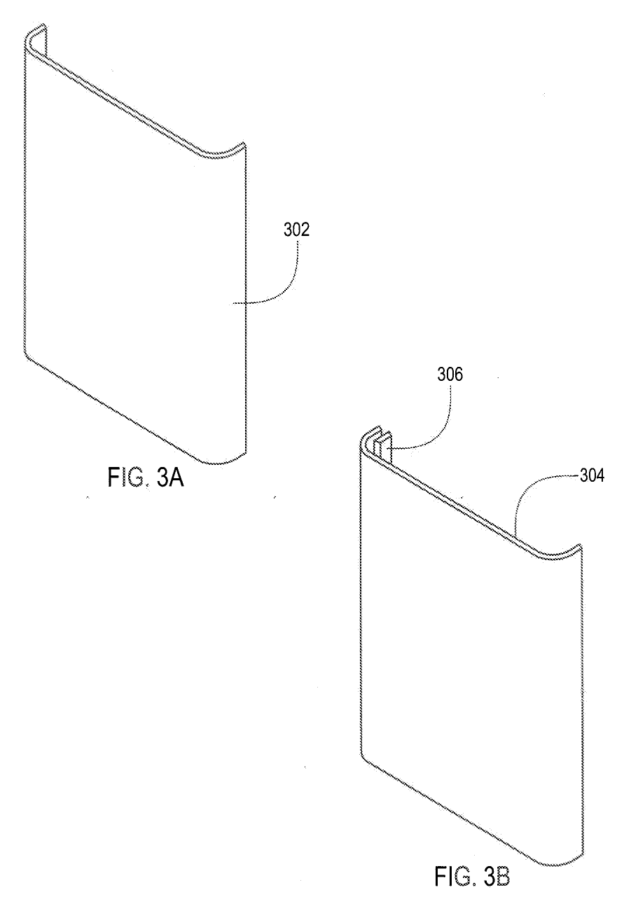

In some embodiments, the enclosure sleeve may not be machined as a whole but is instead machined as two separate halves that are subsequently brought together to form an enclosure sleeve similar to the enclosure sleeve 200 of FIG. 2. FIG. 3A shows an example of one enclosure sleeve half 302. FIG. 4A shows a top view of the enclosure sleeve half 302. In this example, the cross-section is consistent from top to bottom, but it will be appreciated that enclosure sleeve halves may be machined with variations in the cross-section from top to bottom including variation in wall thickness as well as variation in the cross-sectional shape.

FIG. 3B shows an example of another enclosure sleeve half 304, and FIG. 4B shows the enclosure sleeve half 304 in cross-section. This enclosure sleeve half 304 is a mate to the enclosure sleeve half 302 of FIGS. 3A and 4A. A tab 306 is present on each vertical edge as oriented in the example of FIG. 3B. This tab 306 provides a supporting surface for the abutment of the inner side of the vertical edge of the enclosure sleeve half 302 to the vertical edge of the enclosure sleeve half 304. Thus, when laser seam welding is applied to the interfacing edges of the two halves 302, 304 to fix the two halves together to form the complete enclosure sleeve, the tab 306 supports that interface of the two edges during the weld and thereafter. This tab 306 also prevents the laser beam and melted titanium from entering the interior of the sleeve being formed by the two halves 302, 304. The tab 306 may include a radiused junction so as to be a closely matched negative of the radiused edge 308 of the enclosure sleeve half 302.

The tab 306 of this embodiment is shown as ending prior to reaching the top edge of the half 302. This allows space for a top cap discussed below to be seated into the top of the enclosure, sleeve above the tab 306. However, in other embodiments the tap 306 may extend to the top edge of the half 302. In that case a top cap may have a notch that accepts the tab 306 as the top cap is being seated into the top of the enclosure sleeve.

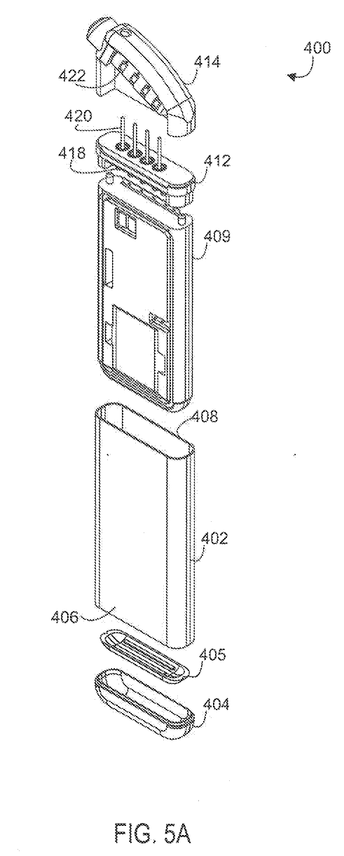

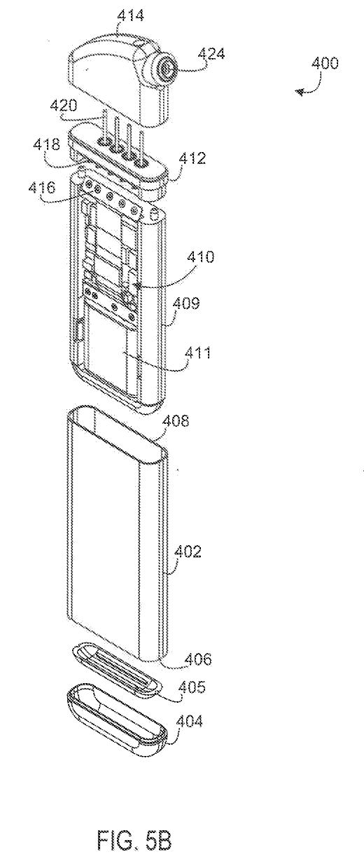

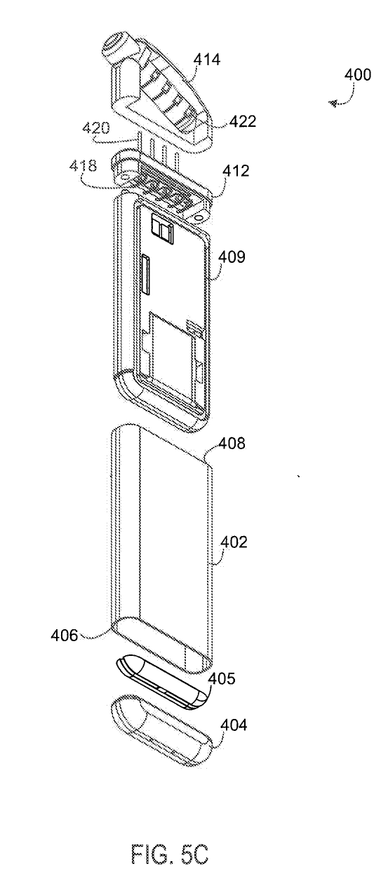

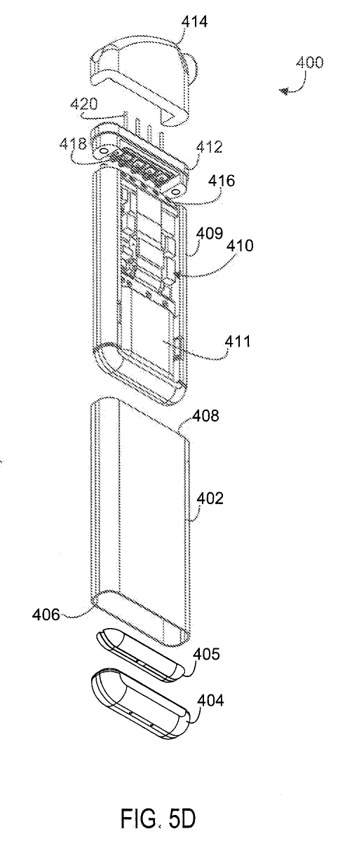

FIGS. 5A-5D show exploded perspective views of an implantable medical device 400 that includes a machined enclosure. A machined enclosure sleeve 402 receives one or more circuit boards 410 that may include features such as a pulse generator for therapy stimulation, sensing circuitry for measuring physiological parameters, telemetry for communication with external devices, a power source, and a recharge circuit. The circuit board 410 of this example includes a flex circuit 416 that extends from the circuit board and carries stimulation and/or sensing signals between the circuitry and a feedthrough block 418 of a top cap 412 which passes the signals via pins 420 to a connector block module 414. The circuit board 410 and an associated battery 411 reside within a polymer chassis 409 in this particular example. The chassis 409 fits snugly within the sleeve 402.

The top cap 412 is attached such as by a laser seam weld to a top edge 408 of the enclosure sleeve 402 to provide a sealed edge. The top cap 412 may be constructed of the same or different material than the enclosure sleeve 402. In this example, the top cap 412 includes the feedthrough block 418 from which the connector pins 420 extend to reach the lead connections 422 of the connector block module 414. For the top cap 412 as shown in FIGS. 5A-5D, this geometry may be machined using a milling process or other applicable machining techniques.

The connector block module 414 mounts to the top of the top cap 412. The top cap 412 may include barbs, pins, or other fasteners that engage receiving features on the bottom of the connector block module 414 to properly position and fix the connector block module 414 in place. The connector block module 414 may include ports that receive the connector pins 420 of the feedthrough block 418 and channel them to connectors 422 that are positioned within channel(s) 424. The channel(s) 424 receive leads that have connectors that mate to the connectors 422 and establish electrical continuity with the connector pins of the feedthrough block 418. One side of the connector block module 414 is shown transparently in FIGS. 5A, 5C, and 5D for purposes of illustrating the channel(s) 424 and connectors 422.

The connector block module 414 may be of a conventional polymer construction. However, the milling process allows the sleeve 402 to be significantly narrower than conventional IMD casings such that the connector block module 414 may also be significantly narrower. To the extent the connector block module 414 may be made so narrow that using conventional attachment features to the top cap 412 become unfeasible, the connector block module 414 may be encased by a metal, such as titanium, and that connector block encasement may be, welded to the top cap 412 to provide a hermetic seal.

A bottom cap 404 is attached such as by a laser seam weld to a bottom edge 406 of the enclosure sleeve 402 to provide a sealed edge. As with the top cap 412, the bottom cap 404 may also be made of the same or different material than the enclosure sleeve 402, and may also be made of the same or different material than the top cap 412. The bottom cap 404 as shown has a bowl or canoe shape. This shape allows a desiccant 405 to be included in the bottom cap 404 and reside beneath the chassis 409 once the IMD 400 is assembled. For the bottom cap 404 as shown in FIGS. 5A-5D, this geometry may be machined using a milling process or other applicable machining techniques.

The desiccant 405 may also serve as a bumper between the chassis 409 and the bottom cap 404 for embodiments where the chassis 409 slides into position within the enclosure sleeve 402 and is held in place at least partially by contact with the bottom cap 404. However, in other embodiments, the desiccant 405 may be positioned elsewhere, such as in a pocket within the chasses 409 and in that case a separate bumper may be placed within the bottom cap 404. In other embodiments, where the chassis 409 is installed within a connector sleeve half so that sliding the chassis 409 within a complete connector sleeve 402 is not performed, the chassis 409 may be glued to the connector sleeve half to hold the chassis 409 in place and a bumper may be omitted particularly where the desiccant 405 is positioned within the chassis 409.

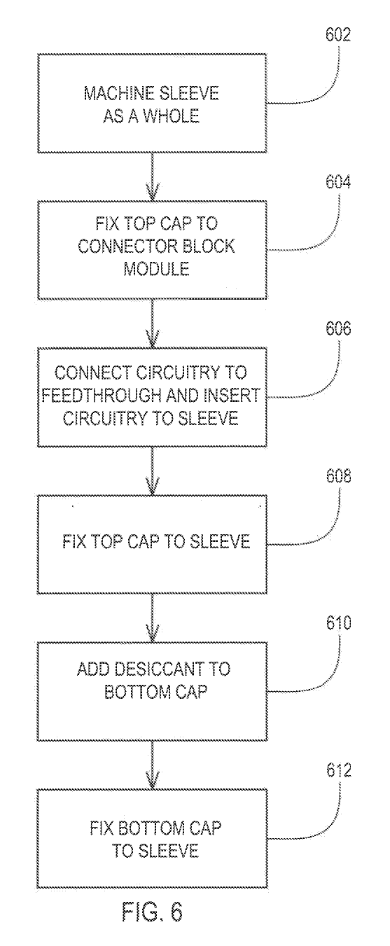

FIG. 6 shows one example of a manufacturing process for an implantable medical device with a machined enclosure. The process begins by machining an enclosure sleeve as a whole, such as that shown in FIG. 2, at a machining step 602. The enclosure sleeve may be machined as a whole by using any of the workpieces and machining processes previously discussed.

The top cap may be fixed to the connector block module by welding or other suitable means of attachment dependent upon the manner of construction of the connector block module as discussed above at a welding step 604. The electrical pins of the feedthrough block of the top cap are routed into the connector block module to make electrical contact with electrical connectors of the connector block module.

Once the top cap and connector block module are joined, the circuitry is connected to the feedthrough of the top cap and the circuitry is loaded into the sleeve at an insertion step 606. At this point, the top cap may then be attached to the sleeve, at an attachment step 608. The top cap may be laser seam or otherwise welded at the top edge of the sleeve.

At this point, a desiccant may be placed into the resting place formed in the bottom cap at a desiccant step 610. By completing the top construction before adding the desiccant and bottom cap, the addition of the desiccant can be delayed until the only remaining step is to add the bottom cap. In this manner, the desiccant is exposed to the ambient conditions for only a short time prior to the interior of the enclosure sleeve being isolated from the exterior. This preserves the effectiveness of the desiccant.

The bottom cap including the desiccant is then fixed to the enclosure sleeve via a laser seam or other weld at a welding step 612. At this point, the enclosure sleeve is sealed and the desiccant is exposed to only the moisture that is already within the enclosure sleeve.

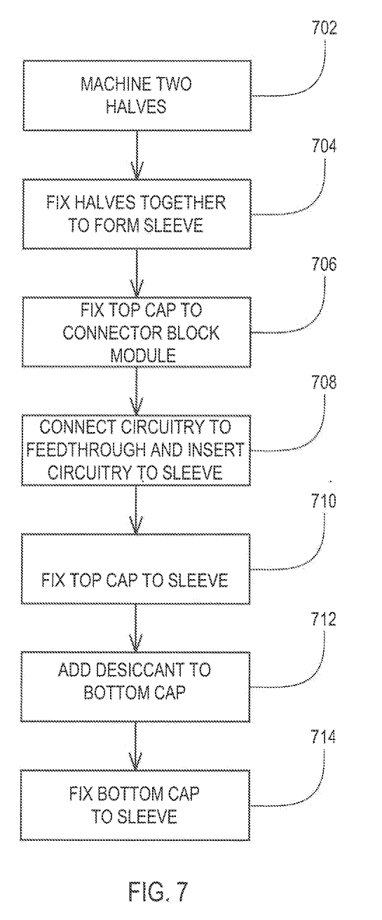

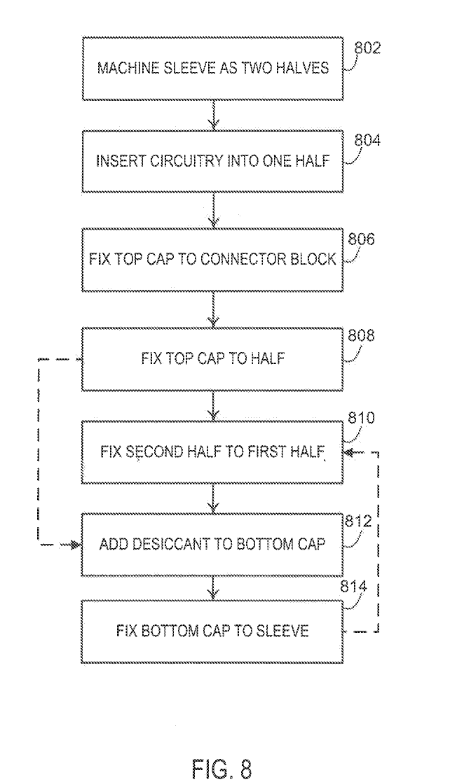

FIG. 7 shows another example of a manufacturing process for an implantable medical device with a machined enclosure. The process begins by machining an enclosure sleeve as two separate halves, such as those shown in FIGS. 3A and 3B, at a machining step 702. The enclosure sleeve halves may be machined using any of the workpieces and machining processes previously discussed.

Once the two complementary enclosure sleeve halves are complete, the two halves may be fixed together to form the enclosure sleeve at a welding step 704.

The top cap may be fixed to the connector block module at a connection step 706, where this connection may involve barbs, adhesives, and other conventional forms of connecting the connector block module or where the connector block module is encased in a metal such as titanium, the connection may be a weld. Once the top cap is joined to the connector block module, the circuitry is connected to the feedthrough of the top cap and the circuitry is loaded into the sleeve at an insertion step 708. The top cap may be attached to the enclosure sleeve at a welding step 710.

At this point, a desiccant may be placed into the bottom cap at a desiccant step 712. As with the process of FIG. 6, by completing the top construction before adding the bottom cap, the addition of the desiccant can be delayed until the only remaining step is to add the bottom cap. In this manner, the desiccant is exposed to the ambient conditions for only a short time prior to the interior of the enclosure sleeve being isolated from the exterior. This preserves the effectiveness of the desiccant.

The bottom cap is then fixed to the enclosure sleeve at a welding step 714. At this point, the enclosure sleeve is sealed and the desiccant is exposed to only the moisture that is already within the enclosure sleeve.

FIG. 8 shows another example of a manufacturing process for an implantable medical device with a machined enclosure. The process begins by machining an enclosure sleeve as two separate halves, such as those shown in FIGS. 3A and 3B, at a machining step 802. The enclosure sleeve halves may be machined using any of the workpieces and machining processes previously discussed.

Once at least one of the two complementary enclosure sleeve halves is complete, the circuitry may be placed into one of the halves at an insertion step 804. In conjunction with inserting the circuitry, the top cap may be fixed to the connector block module at a connection step 806, where this connection may involve barbs, adhesives, and other conventional forms of connecting the connector block module or where the connector block module is encased in a metal such as titanium, the connection may be a weld. Once the top cap is joined to the connector block module, the top cap may be attached to the enclosure sleeve half, with the electrical connections to the circuitry being completed, at an attachment step 808.

At this point, FIG. 8 presents alternative paths. In one example, the second half of the enclosure sleeve may be fixed to the first half to complete the sleeve at a welding step 810. A desiccant may then be placed into the bottom cap at a desiccant step 812, and the bottom cap is then fixed to the enclosure sleeve at a welding step 814. In another example, after attaching the top cap to the first half, the desiccant may then be placed into the bottom cap at a desiccant step 812, and the bottom cap is then fixed to the enclosure sleeve at a welding step 814. The second half of the enclosure sleeve is then attached to the first half at the welding step 810.

While the preceding examples of manufacturing involve the creation of an enclosure sleeve, other examples of manufacturing an implantable medical device with a machined enclosure are also applicable. For instance, rather than creating an enclosure sleeve as a whole or as two joined halves with top and bottom caps, two conventional halves may be milled rather than stamped. Circuitry, a connector block module, and desiccant may then be added in the conventional way.

While embodiments have been particularly shown and described, it will be understood by those skilled in the art that various other changes in the form and details may be made therein without departing from the spirit and scope of the invention.

What is claimed is:

1. A method of creating an implantable medical device, comprising:
machining biocompatible material to produce an enclosure sleeve having an open top and open bottom;
installing circuitry within the enclosure sleeve;
attaching a top cap onto the enclosure sleeve to close the open top;
mounting a connector block module to the top cap; and
attaching a bottom cap onto the enclosure sleeve to close the open bottom.

2. The method of claim 1, wherein attaching the top cap and attaching the bottom cap comprises welding the top and bottom caps.

3. The method of claim 1, wherein the material is tubular stock.

4. The method of claim 1, wherein machining the material comprises electric discharge machining of the material.

5. The method of claim 4, wherein wire electric discharge machining of the material produces an outside geometry and an inside geometry of the enclosure sleeve.

6. The method of claim 4, wherein wire electric discharge machining of the material produces an inside geometry of the enclosure sleeve and milling of the material produces an outside geometry of the enclosure sleeve.

7. The method of claim 1, wherein the material has a hardness of grade five titanium or harder.

8. The method of claim 7, wherein the material is titanium of grade five or harder.

9. A method of creating an implantable medical device, comprising:
machining biocompatible material to produce two enclosure halves;
welding the two enclosure halves together to produce an enclosure;
installing circuitry within the enclosure; and
mounting a connector block module to the enclosure.

10. The method of claim 9, wherein the enclosure forms an enclosure sleeve that has an open top and open bottom, the method further comprising:
attaching a top cap onto the enclosure sleeve to close the open top; and
attaching a bottom cap onto the enclosure sleeve to close the open bottom.

11. The method of claim 10, wherein attaching the top cap and attaching the bottom cap comprises welding the top and bottom caps.

12. The method of claim 9, wherein the material is bar stock.

13. The method of claim 9, wherein machining of the material comprises electric discharge machining.

14. The method of claim 13, wherein wire electric discharge machining produces an outside geometry and an inside geometry of the two enclosure halves.

15. The method of claim 13, wherein wire electric discharge machining of the material produces an inside geometry of the two enclosure halves and milling produces an outside geometry of the two enclosure halves.

16. The method of claim 15, further comprising annealing the two enclosure halves.

17. The method of claim 9, wherein the material has a hardness of grade five titanium or harder.

18. The method of claim 17, wherein the material is titanium of grade five or harder.

19. An implantable medical device, comprising:
an enclosure that has a geometry that is machined from biocompatible material;
circuitry within the enclosure; and
a connector block module fixed to a top of the enclosure.

20. The implantable medical device of claim 19, wherein the enclosure comprises an enclosure sleeve, a top cap, and a bottom cap, wherein the connector block module is fixed to the top cap.

21. The implantable medical device of claim 20, wherein the top cap and bottom cap are welded to the enclosure sleeve.

22. The implantable medical device of claim 19, wherein the material is bar stock.

23. The implantable medical device of claim 19, wherein the material is tubular stock.

24. The implantable medical device of claim 20, wherein the enclosure sleeve is formed of two halves that are electric discharge machined from material.

25. The implantable medical device of claim 24, wherein an inside geometry and an outside geometry of the two halves are wire electric discharge machined.

26. The implantable medical device of claim 24, wherein an inside geometry of the two halves is wire electric discharge machined and an outside geometry of the two halves is milled, and wherein the two halves are annealed.

27. The implantable medical device of claim 20, wherein an inside geometry and an outside geometry of the enclosure are wire electric discharge machined.

28. The implantable medical device of claim 20, wherein an inside geometry of the enclosure is wire electric discharge machined and an outside geometry of the enclosure is milled, and wherein the enclosure sleeve is annealed.

29. The implantable medical device of claim 18, wherein the material has a hardness of grade five titanium or harder.

30. The implantable medical device of claim 29, wherein the material is titanium of grade five or harder.
